(19) United States
(12) Patent Application Publication
(10) Pub. No.: US 2022/0015468 A1
(43) Pub. Date: Jan. 20, 2022

(54) PROTECTIVE FACE MASK FOR INVASIVE MEDICAL PROCEDURES THROUGH MOUTH OR NOSE

(71) Applicant: Ehk Medical, LLC, Akron, OH (US)

(72) Inventors: Eric A. Espinal, Akron, OH (US); Robert Alan Hagarty, Akron, OH (US); Phillip Labib Khalil, Medina, OH (US)

(73) Assignee: Ehk Medical, LLC, Akron, OH (US)

(21) Appl. No.: 16/933,352

(22) Filed: Jul. 20, 2020

(51) Int. Cl. A41D 13/11; A61M 16/20; A61M 39/04

(52) U.S. Cl. CPC A41D 13/1138; A61B 1/00147; A61M 39/04; A61M 16/20

(57) ABSTRACT

A medical device for use with an associated tube inserted into a respiratory outlet of a patient is disclosed. The medical device includes a mask with an opening sized to receive and allow movement of the associated tube, and a valve operatively connected with the opening of the mask. The valve includes a main body, and an opening formed in a portion of the main body in which the opening is sized and dimensioned to accommodate the associated tube.

### BACKGROUND

This disclosure relates to a device or apparatus for a protective face mask with a valve to accommodate a surgical (i.e., an endoscopy scope) tubular device. For example, this disclosure describes exemplary embodiments for a valve installed on a protective face mask to accommodate a tubular scope inserted into a patient during a surgical or medical procedure such as, for example, endoscopy procedures. However, it will be appreciated that the disclosed concepts may have usefulness in accommodation of other diagnostic or therapeutic medical devices, such as various types of laryngoscopes bronchoscopes, gastroscopes, endoscopes, cannulas, gastric tubes, feeding tubes, endotracheal tubes, and other therapy delivery tubes.

By way of background, medical procedures, such as laryngoscopy procedures, involve a medical professional inserting a diagnostic device into the mouth or nasal passages (i.e., nostrils) of a patient. However, the patient can expel microbes through the nose or mouth during such procedures. These microbes can become airborne or aerosolized in the air. These particles can be inhaled by the medical professional. Aerosolization of microbes and regional contamination can occur for many hours, thereby exposing anyone who would enter the room after the procedures. Even if not inhaled, infected particles can contaminate the medical professional or others in the environment, which could then contaminate other patients with which they come in contact.

While most healthcare practitioners practice universal precautions and try to avoid contamination with airborne and/or aerosolized microbes, the patient's mouth and nose cannot be covered during the medical procedure, which can result in the medical professional being exposed to microbes and infectious diseases (e.g., by the patient breathing, sneezing, or expelling saliva).

In particular, the global COVID-19 pandemic has emphasized the need to minimize environmental contamination with macroscopic as well as microscopic secretions as well as aerosolization of microbes, which could be lethal to those residing in the healthcare environment (i.e., healthcare workers and other patients) (see, e.g., Cha, {2020}, ‘CDC Says More Than 9,000 Healthcare Workers Had Tested Positive For Coronavirus As Of April 9’, Washington Post, April 14, 2020; and Stobbe, {2020} ‘Health Care Workers are 10%-20% of US Coronavirus Cases’; Associated Press (AP) News, Apr. 14, 2020). In addition, not only might healthcare workers be contaminated, but these infected healthcare workers may contaminate other patients, healthcare workers, or other people they might subsequently come into contact with. While COVID-19 has affected the globe in 2020, a new and augmented approach to disease prevention will be sought in the future.

Based on the foregoing, a solution that covers the patient's mouth and nasal passages, while allowing insertion of a surgical tube into the patient's mouth and nasal passages is desirable. Additionally, a solution that provides protection for medical professionals during the insertion of such a surgical tube is desirable. While applicable to laryngoscopy procedures, such solutions may be applicable to other medical professionals including dentists, bronchoscopists, endoscopists, and other medical professionals who perform procedures requiring close contact to the nasopharynx or oropharynx.

### SUMMARY

In one aspect, a medical device for use with an associated tube inserted into a respiratory outlet of a patient is disclosed. The medical device includes a mask with an opening sized to receive and allow movement of the associated tube, and a valve operatively connected with the opening of the mask. The valve includes a main body, and an opening formed in a portion of the main body in which the opening is sized and dimensioned to accommodate the associated tube.

In another aspect, a medical kit for use with an associated tube inserted into a respiratory outlet of a patient is disclosed. The kit includes a mask with an opening sized to receive and allow movement of the associated tube, a viewing portion secured to a portion of the mask and surrounding the opening thereof, and a valve operatively connected with the opening of the mask. The valve includes a main body, and an opening formed in a portion of the main body. The valve is configured to be inserted into the opening in the mask for attachment thereto.

In yet another aspect, a medical device for use with an associated tube inserted into a respiratory outlet of a patient is disclosed. The medical device includes a mask with an opening sized to receive and allow movement of the associated tube, a viewing portion secured to a portion of the mask and surrounding the opening thereof, and a strip operatively connected with the viewing portion. The strip includes at least one valve formed in a portion thereof. The at least one valve is sized and dimensioned to accommodate the associated tube.

Further scope of the applicability of the present invention will become apparent from the detailed description provided below. It should be understood, however, that the detailed description and specific examples, while indicating preferred embodiments of the invention, are given by way of illustration only, since various changes and modifications within the spirit and scope of the invention will become apparent to those skilled in the art.

### DESCRIPTION OF THE DRAWINGS

The present invention exists in the construction, arrangement, and combination of the various parts of the device, and steps of the method, whereby the objects contemplated are attained as hereinafter more fully set forth, specifically pointed out in the claims, and illustrated in the accompanying drawings in which:

FIG. 1 is a perspective view of an exemplary embodiment of a medical device;

FIG. 2 is a schematic view of a mask of the medical device of FIG. 1;

FIGS. 3A-E are schematic views of a valve of the medical device of FIG. 1;

FIG. 4 is a schematic view of a viewing portion of the medical device of FIG. 1;

FIGS. 5-8 show alternate views of the medical device of FIG. 1; and

FIGS. 9-23 show an alternative embodiment of the medical device of FIG. 1.

### DETAILED DESCRIPTION

Various embodiments of a medical device and associated methods of using or manufacturing the medical device for use with a surgical tube in conjunction with a surgical procedure are disclosed herein. Generally, the medical device includes a protective mask to cover a patient's respiratory outlets (i.e., the nasal passages and mouth), and one or more ports comprising a valve to accommodate a surgical tube for insertion into the nasal passages or mouth of the patient by a medical professional. In certain embodiments, the valve is sized and dimensioned to receive the tube, and includes one or more flaps to seal the valve, and thus seal the patient's mouth and nasal passages during insertion of the surgical tube and after removal of the surgical tube.

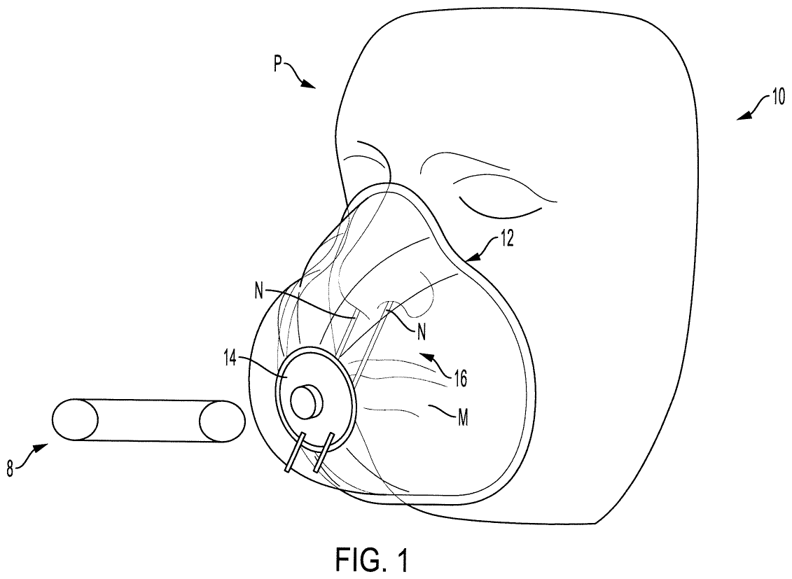

Referring now to the drawings wherein the showings are for purposes of illustrating the exemplary embodiments only and not for purposes of limiting the claimed subject matter, FIG. 1 depicts an exemplary embodiment of a medical device 10 for use with an associated surgical tube 8. The surgical tube 8 is used during surgery, in which the surgical tube 8 is inserted into a nasal passage N (two of which are shown in FIG. 1) or a mouth M of a patient P (e.g., during a laryngoscopy procedure, an endoscopy procedure, and so forth). For example, the surgical tube 8 can comprise an endoscope with a camera mounted thereon to examine the respiratory pathways of the patient P. However, the surgical tube 8 can comprise any suitable tube for insertion into a respiratory entryway (e.g., the nasal passages N or the mouth M) of the patient P, such as oral endoscopy procedures (e.g., bronchoscopy, esophagoscopy, gastroscopy, esophagogastroduodenoscopy, transesophageal echocardiography (TEE), and laryngoscopy) and nasal endoscopy procedures, trans-nasal esophagoscopy, as well as various types of enteric feeding tubes. While applicable to laryngoscopy procedures, such solutions may be applicable to other medical professionals including dentists, bronchoscopists, cardiologists, anesthesiologists, endoscopists, and other medical professionals who perform procedures requiring close contact to the nasopharynx, oropharynx, or mouth.

As shown in FIG. 1, the device 10 includes a mask 12 (which is shown as being transparent in FIG. 1 to allow visualization of other components) configured to cover, enclose, or otherwise surround the nasal passages N and the mouth M of the patient P, a valve 14 attached to a portion of the mask 12 and also configured to the nasal passages N and the mouth M of the patient P, and configured to accommodate the surgical tube 8 through the mask 12, and a viewing portion 16 attached to a portion of the mask 12 and configured to allow a medical professional to visualize the surgical tube 8 after insertion through the valve 14. The device 10 can be used for suitable patients, such as patients already not wearing protective masks for the surgical procedures, and the device 10 can be removed from the patient P after the surgical procedure. Each of the mask 12, the valve 14, and the viewing portion 16 are described in more detail below.

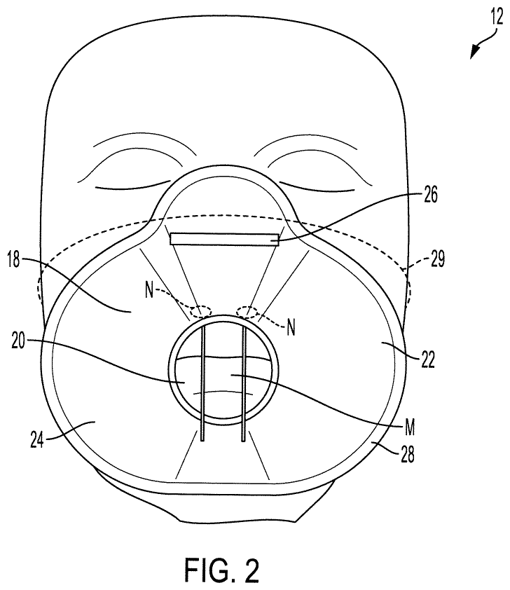

FIG. 2 shows an example of the mask 12. The mask 12 can include a main mask body 18 with a mask opening 20 through which the valve 14 can be installed. The mask 12 can include a cover side 22 (i.e., a “back” side) which covers the nasal passages N and the mouth M, and an opposing exposed side 24 (i.e., a “front side”) which is exposed to the ambient environment. The mask 12 can include a shaping portion 26 located on a portion of the mask 12 overlaying a bridge of the nose of the patient P. The shaping portion 26 can comprise a bendable metallic portion (e.g., aluminum) which can be manipulated by the patient P or the medical professional to form a tighter seal around the patient's nose. A rim 28 of the mask 12 provides a seal around the perimeter of the mask 12 when the mask 12 is attached to the patient P via one or more straps 29 (e.g., elastic straps) configured to wrap around a head of the patient P. The mask 12 can be made of a substantially pliable material (e.g., plastics such as polyurethane, polypropylene, and polyester) that filter unwanted microbes from the patient's breath into the ambient environment. That is, the mask 12 is pliable enough to be manipulated, but has some rigidity to avoid tearing. As shown in FIG. 2, the mask 12 has an elliptical or circular shape; although any planar shape can be suitable. The mask 12 can be any suitable mask, including a commercially-available mask, such as an N95 mask (available from The 3M Company, Maplewood, Minn., USA, Honeywell, Charlotte, N.C., USA or Kimberly-Clark Professional Corp., Irving, Tex., USA).

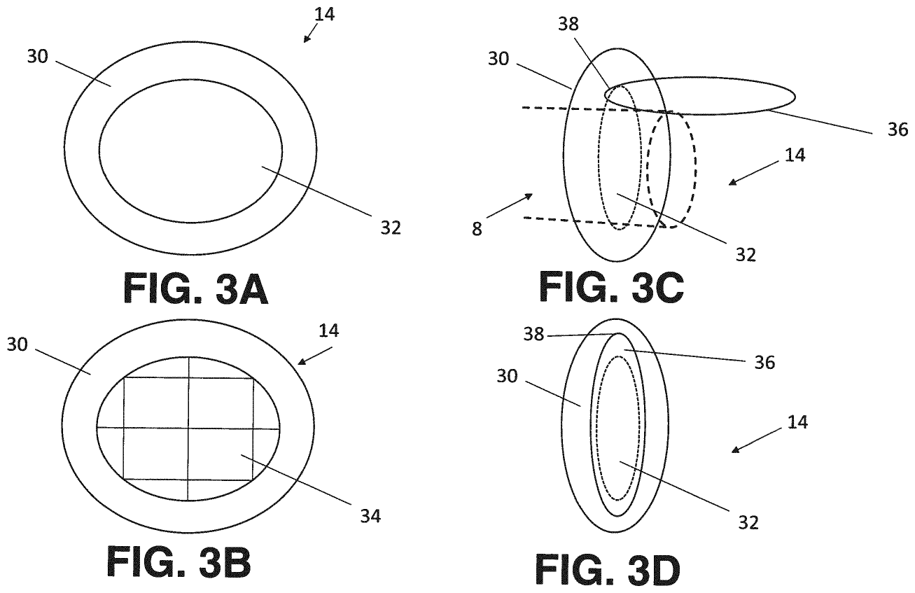

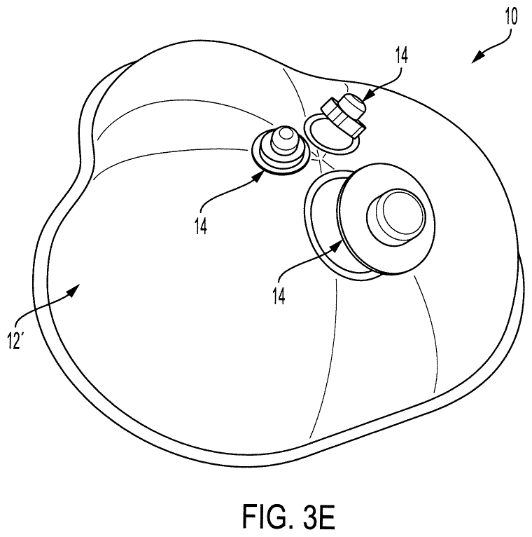

FIGS. 3A-E show examples of the valve 14. Starting at FIG. 3A, the valve 14 includes a main valve body 30 with a valve opening 32. The valve opening 32 is sized and dimensioned to receive and accommodate the surgical tube 8 (FIG. 1). Surgical tubes used in nasal or oral insertion operations can have a diameter of approximately 4 mm; however, it will be appreciated that the surgical tube 8 can have a diameter of any suitable size. Thus, a diameter of the valve opening 32 can have similar dimensions as the diameter of the surgical tube 8 to receive and accommodate the surgical tube 8, which can also advantageously prevent leaking of air or saliva particles of the patient P from passing through the valve 14 and to the medical professional. In some examples, the valve 14 can include an adapter (not shown) to allow the valve 14 to fit any-sized surgical tube 8. In some embodiments, the valve 14 is configured to accommodate a range of sizes of surgical tubes 8 (e.g., surgical tubes for nasal endoscopes, laryngoscopes, trans-nasal esophagoscopes, bronchoscope, esophagogastroduodenoscopes (EGD), transesophageal echocardiography (TEE) procedures, and so forth).

In an alternate embodiment, as shown in FIG. 3B, the valve 14 includes a knock-out portion or element 34 centrally located on the main valve body 30. In lieu of providing a predefined opening 32, the knock-out portion 34 can be made of a substantially thin, flexible material (e.g., a thin, mesh-like material). When the medical device 10 is fitted on the surgical tube 8, an end of the surgical tube 8 can perforate (i.e., knock out) a portion of the knock-out portion 34 to create the valve opening 32. Advantageously, the knock-out portion 34 allows the valve opening 32 to be formed with substantially the exact size of the diameter of the surgical tube 8, which can prevent fluid or microbes from entering or exiting the from the nasal passages N or the mouth M of the patient P (and thus preventing aerosolized particles that can be inhaled by the medical professional) during the surgical procedure through the valve opening 32. Alternatively, the knock-out portion 34 can include a series of perforated portions (not shown) corresponding to the potential sizes of the surgical tube 8. That is, a first perforated portion can be sized to 5 mm, a second perforated portion can be sized to 6 mm, and so forth. The end of the surgical tube 8 can punch-out the corresponding perforated portion upon installation of the medical device 10 onto the surgical tube 8.

As shown in FIG. 3C, the valve 14 also includes a suitable mechanism that is sized and dimensioned to cover the valve opening 32, such as a flap 36. The flap 36 is disposed on the side of the main valve body 30 that faces the patient P. Advantageously, the flap 36 covers the valve opening 32 after removal of the surgical tube 8 to provide seal around the nasal cavities N and the mouth M of the patient P, as shown in FIG. 3D. The flap 36 can have any suitable dimensions to cover the valve opening 32 (e.g., approximately 6 mm to approximately 13 mm to cover any size of the valve opening ranging from approximately 5 mm to approximately 12 mm).

The flap 36 can be attached to the main valve body 30 with a hinged connection or joint 38, so that the flap 36 is resiliently biased relative to the main valve body 30. The hinged connection 38 allows the flap 36 to be pushed away from the valve opening 32 when the surgical tube 8 is inserted through the valve opening 32 (i.e., an open state). As shown in FIG. 3C, the flap 36 can be biased upwards relative to the valve main body 30 by the surgical tube 8 when the surgical tube 8 is inserted through the valve opening 32. As shown in FIG. 3D, upon removal of the surgical tube 8 (not shown in FIG. 3D), the flap 36 is no longer biased upward, and can move towards a natural collapsed state covering the valve opening 32 (i.e., a closed state).

As shown in FIGS. 3A-D, the valve 14 has a circular shape, although any planar shape can be suitable. The valve 14 can be made of a substantially rigid material (e.g., plastic). That is, the valve 14 is flexible enough to be manipulated, but rigid enough to avoid tearing and to allow movement of the surgical tube 8 therethrough. The valve 14 can be any suitable valve, including a commercially-available valve.

In addition, although the device 10 is described herein as including only one valve 14, the device 10 can include any suitable number of valves 14 (e.g., two or more). FIG. 3E shows another embodiment of the mask 12′ that includes three valves 14, two of which cover individual nasal passages N, and a third which covers the mouth M of the patient P.

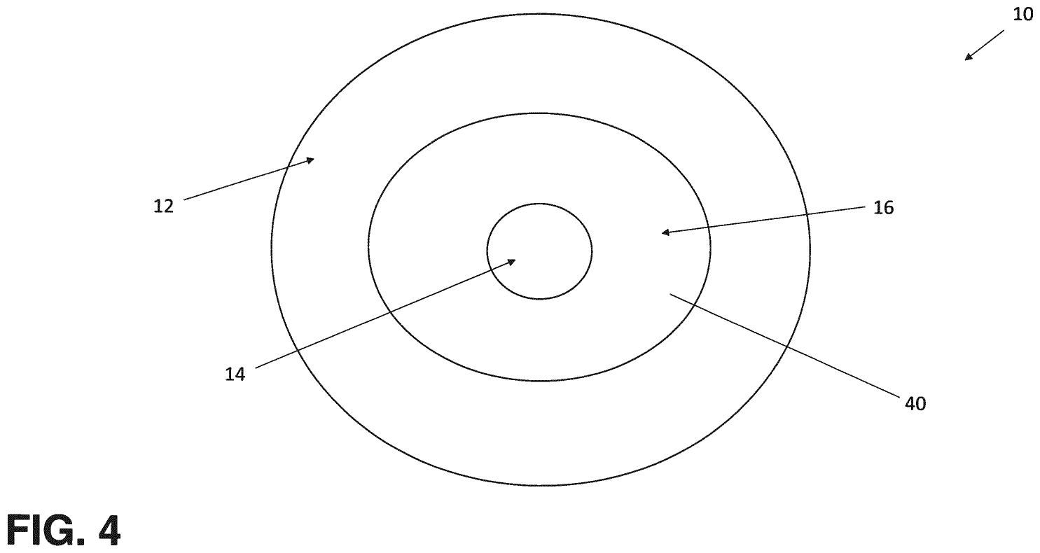

FIG. 4 shows an example of the viewing portion 16. As shown in FIG. 4, the viewing portion 16 includes a main viewing portion body 40 is attached to the exposed side 24 of the mask 12 (e.g., with stitching, adhesives, or any other suitable attachment mechanism) and surrounds the valve 14.

The viewing portion 16 can be made of a substantially pliable material (e.g., plastic). That is, the viewing portion 16 is pliable enough to be manipulated, but has enough rigidity to avoid tearing or be impermeable to air or fluid that can enter the surgical environment of the surgical tube 8. Existing masks can be made from occlusive materials, preventing a medical professional from visualizing the patient's nostrils N and/or mouth M. Advantageously, the viewing portion 16 can be made of a transparent material (e.g., plastic) to allow the medical professional to visualize the surgical tube 8 within an area defined by the mask 12 that covers a portion of the patient's face. The medical professional can then adjust a trajectory of the surgical tube 8 in order to insert the surgical tube 8 into one of the nasal passages N or the mouth M of the patient. As shown in FIG. 4, the viewing portion 16 has a circular shape; although any shape can be suitable. The viewing portion 16 can be a component of the commercially-available mask. In some embodiments, the viewing portion 16 can be omitted, and the main mask body 20 can be made of a transparent material.

In some embodiments, the medical device 10 can comprise a kit that includes the mask 12 and one or more valves 14. The valve(s) 14 can be inserted into the openings 20 of the mask 12. The opening 20 can be formed in the mask 12 by puncturing the main mask body 18. Alternatively, the mask opening 20 can be pre-formed in the main mask body 18.

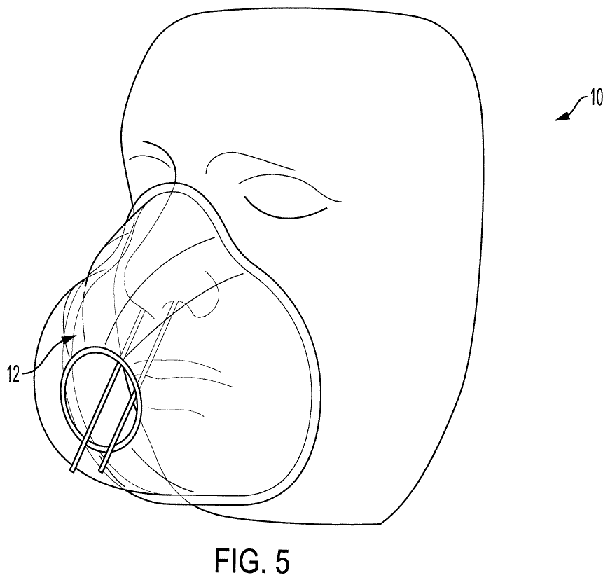

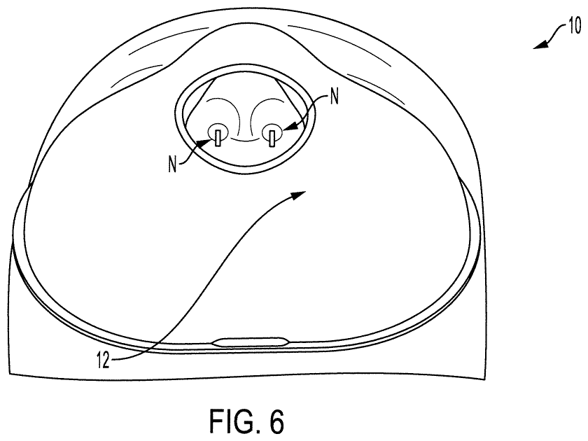

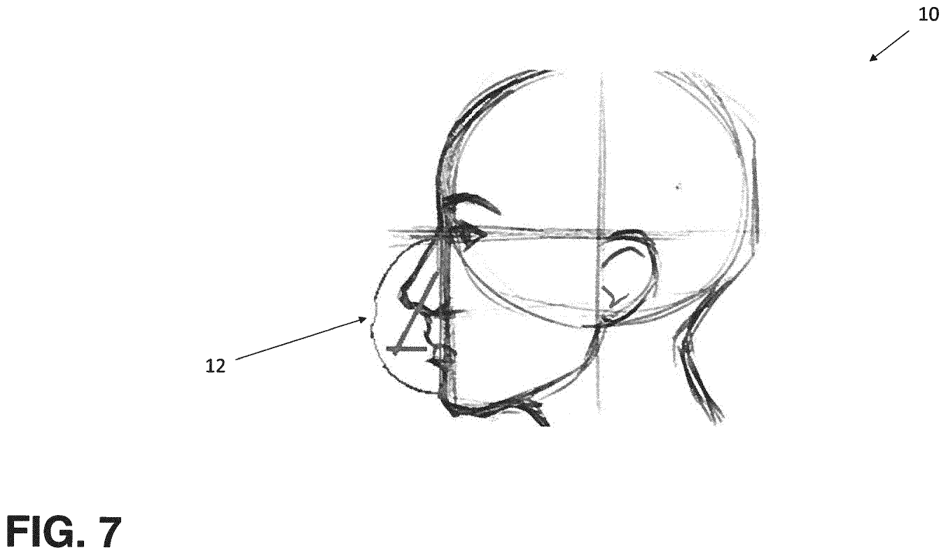

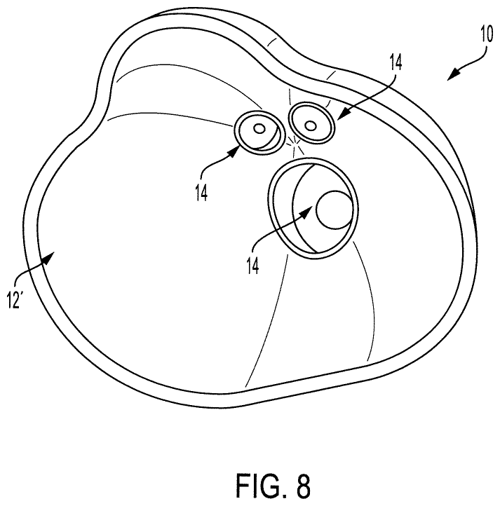

FIGS. 5-8 show alternate views of the medical device 10. FIGS. 5-7 show the medical device 10 with the mask 12, and FIG. 8 shows the medical device 10 with the mask 12′.

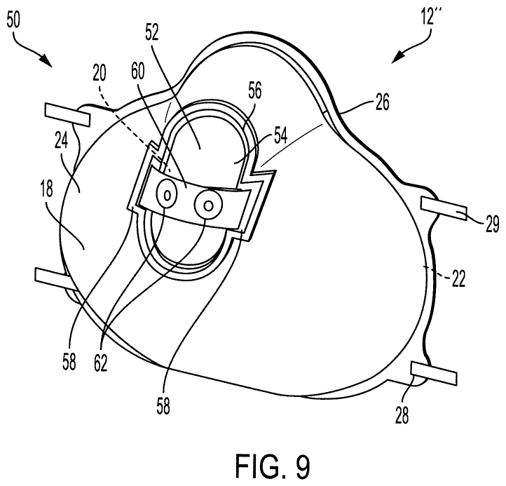

FIG. 9 shows another embodiment of a medical device 50. The medical device 50 comprises substantially the same components as the medical device 10 of FIGS. 1-8, except as described henceforth. As shown in FIG. 9, the medical device 10 includes a different embodiment of the mask 12″ that includes the mask opening 20. A viewing portion 52 is installed over the mask opening 20. The viewing portion 52 is elliptically-shaped; although any planar shape is suitable (and substantially matches the shape of the mask opening 20). The viewing portion 52 includes a viewing panel 54 made of a transparent material (e.g., plastic) for viewing the nasal passages N and/or the mouth M. A rim 56 surrounds a perimeter of the viewing panel 54 and is attached to the main mask body 18 by any suitable means (e.g., stitching, adhesives, and so forth). The rim 56 includes one or more slot portions 58 (two of which are shown in FIG. 9, although any suitable number of slot portions can be included). A strip 60 is inserted at its ends into the slot portions 58. The strip 60 includes at least one valve 62 (two of which are shown in FIG. 9, although any suitable number of valves can be included) that overlays the nasal passages N or the mouth M of the patient P. For example, the strip 60 can include two valves 62 overlaying each of the nasal passages N, one valve 62 overlaying the mouth M, or any suitable combination thereof. The at least one valve 62 is sized and dimensioned to receive and accommodate the surgical tube 8.

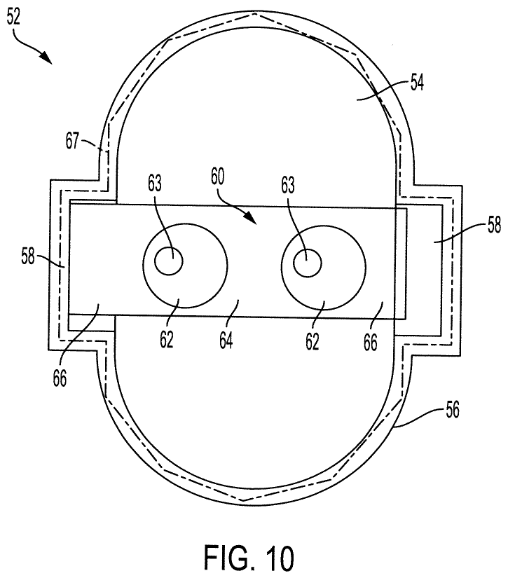

FIG. 10 shows a more detailed view of the viewing portion 52 and the strip 60. As shown in FIG. 10, the strip 60 has a rectangular shape; although any planar shape can be suitable. The strip 60 comprises a main strip body 64, through which the valves 62 are formed. The main strip body 64 can comprise a relatively thin section of pliable, yet stable, material. The main body strip 64 secures the valves 62. The valves 62 can be made from an expanding sponge-like material. This sponge-like material is configured to form a barrier around the surgical tube 8 when inserted through the valve 62, and wipes the surgical tube 8 of fluid when the surgical tube 8 is removed from the valve 62. In some examples, the viewing panel 54 can secure the strip 60 to the mask 12″.

As shown in FIG. 10, the valves 62 comprise indentations in the main strip body 64 extending from a top planar surface of the main strip body 64 to a bottom planar surface of the main strip body that overlays the viewing panel 54. Ends 66 of the strip 60 are inserted into corresponding slot portions 58 of the rim 56 of the viewing portion 52 and are held in a friction-fit in the slot portions 58. Once installed, the valves 62 overlay a corresponding number of openings 63 (shown in FIG. 10 as being overlaid by the valves 62) formed in the viewing panel 54, thereby allowing insertion and removal of the surgical tube 8. In one example, the ends 66 of the strip 60 are inserted into the slot portions 58 so that the strip 60 is disposed between the viewing panel 54 and the mask 12″ (i.e., the strip 60 is inside of the viewing portion 52). In another, example the ends 66 of the strip 60 are inserted into the slot portions 58 so that the strip 60 is disposed on the viewing panel 54 (i.e., the strip 60 is outside of the viewing portion 52). The strip 60 can be moved in a left-right direction or an up-down direction in the slot portions 58 by the user to accommodate the surgical tube 8. FIG. 10 also shows that the rim 56 of the viewing portion 52 includes a groove 67 extending around the perimeter of the rim 56.

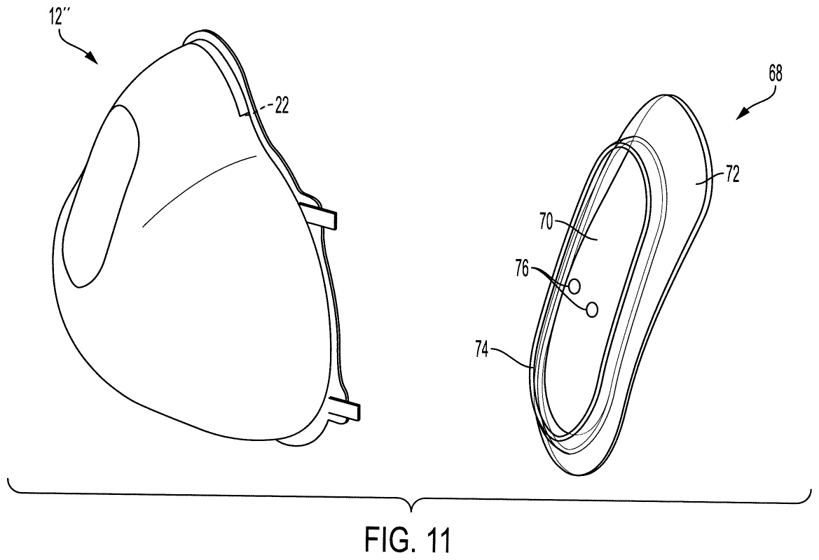

FIG. 11 shows a backing portion 68 attached to the cover side 22 of the mask 12 (note that the mask 12″ is transparently shown in FIG. 11 to allow visualization of the backing portion 68). As shown in FIG. 11, the backing portion 68 has an elliptical shape; although any planar shape can be suitable. The backing portion 68 includes a viewing window 70 and a rim 72. The viewing window 70 and the rim 72 can be made of any suitable plastic material, with the viewing window being made of a transparent plastic material. In some embodiments, the viewing window 70 may comprise an opening instead of being made of a plastic material. The rim 72 includes a protruding portion 74 protruding from the rim 72 and extending around a perimeter of the viewing panel 54. The viewing window 70 also includes a number of openings 76 (two of which are shown in FIG. 11) configured to align with the valves 62 (not shown in FIG. 11) and the openings 63 of the viewing portion 54 (not shown in FIG. 11) to receive and accommodate the surgical tube 8. The number of openings 76 corresponds to the number of valves 62 and the number of openings 63 of the viewing portion 54.

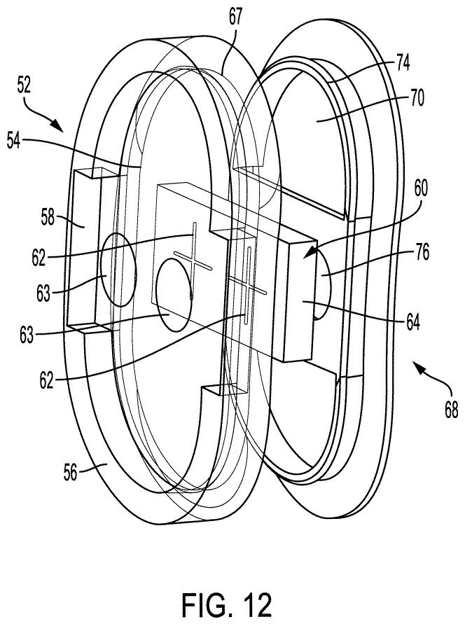

FIG. 12 shows another view of the viewing portion 52, the strip 60, and the backing portion 68 positioned relative to each other.

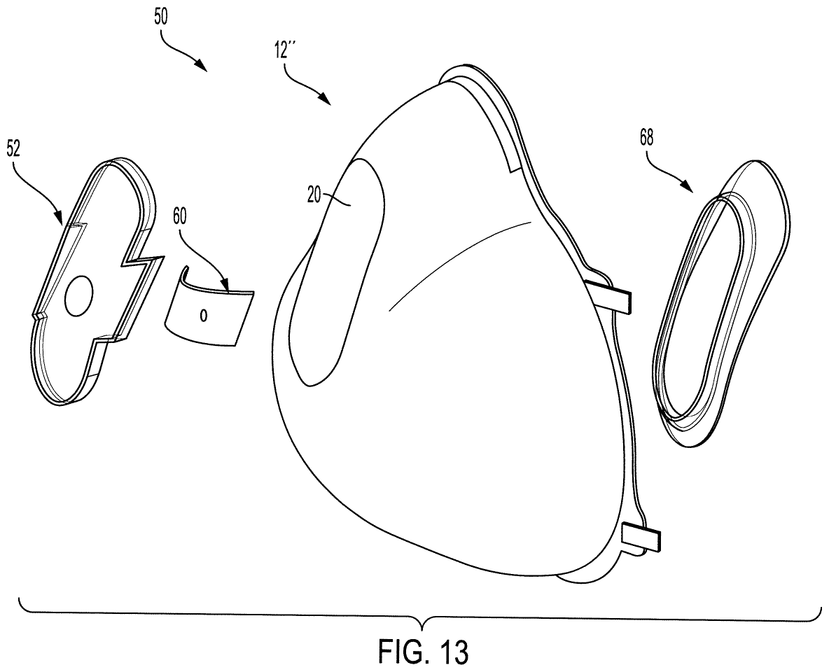

With continuing reference to FIGS. 9-12, FIG. 13 shows an assembly view of the medical device 50. The backing portion 68 is disposed on the cover side 22 of the mask 12″, and is installed onto the mask opening 20. To do so, the protruding portion 74 of the rim 72 is passed through mask opening 20 until the rim 72 contacts the cover side 22 of the mask 12″. The protruding portion 74 of the rim 72 extends past the main mask body 18. The protruding portion 74 and the remainder of the rim 72 form an encapsulated fit against the main mask body 18 to secure the backing portion 68 to the mask 12 (i.e., the mask 12 is wedged into a groove (not shown) formed by the union of the protruding portion 74 and the rim 72).

The viewing portion 52 is then installed onto the mask 12. The groove 67 of the rim 56 of the viewing portion 52 is fit onto the protruding portion 74 of the rim 72 (e.g., in a male-female friction fit). The rim 56 of the viewing portion 52 is slid onto the protruding portion 74 of the rim 72 until the viewing portion 52 contacts the exposed side 24 of the mask 12″. The protruding portion 74 and the groove 67 have suitable dimensions so that the protruding portion 74 extends completely into the groove 67 when the viewing portion 52 contacts the exposed side 24 of the mask 12″. Once the viewing portion 52 is fit onto the mask 12″, the viewing panel 54 of the viewing portion 52 is aligned with the viewing window 70 of the backing portion 68. Since both the viewing panel 54 and the viewing window 70 are both made of transparent materials, the medical professional can visualize the nasal passages N and/or the mouth M of the patient P. In some embodiments, the groove 67 and the protruding portion 74 can be omitted, and the viewing portion 52 can be secured to the mask 12 with stitching, adhesives, or any other suitable attachment mechanism.

The strip 60 is then attached to the viewing portion 52 by inserting the ends 66 of the strip 60 into the corresponding slot portions 58 of the viewing portion 52. Once the strip 60 is installed, the surgical tube 8 can be inserted through one of the valves 62 (and the openings 63 and 76) towards the nasal passages N and/or the mouth of the patient P.

In some embodiments, the medical device 50 can comprise a medical kit for assembly by a user. For example, the kit can include the mask 12, the viewing portion 52, the strip 60, and the backing portion 68 assembled as described herein to form the medical device 50. In another example, the kit can include an assembled mask 12, viewing portion 52, and backing portion 68, and the strip 60 can be inserted by the user. Once used on the patient P, the medical device 50 can be disposed, thereby preventing exposure of medical professionals and the patient P to microbes.

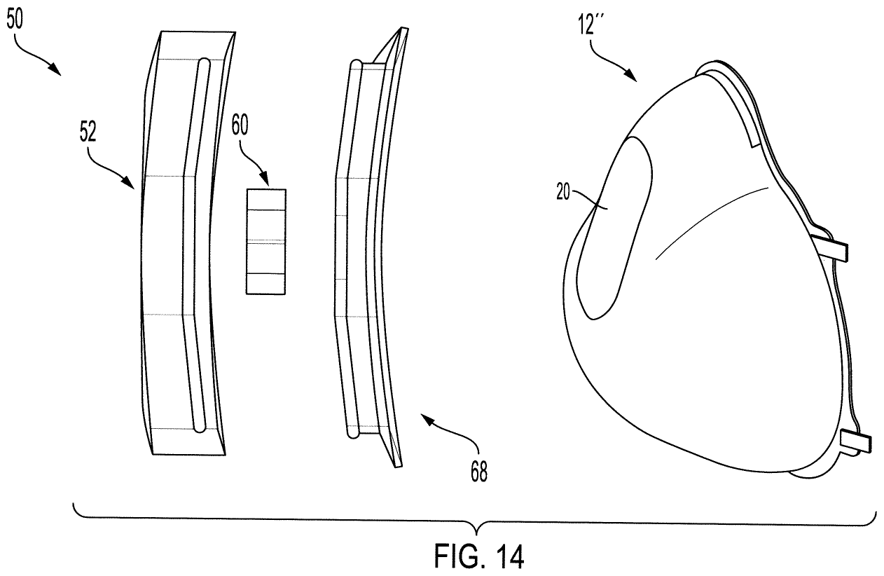

FIG. 14 shows another assembly view of the medical device 50.

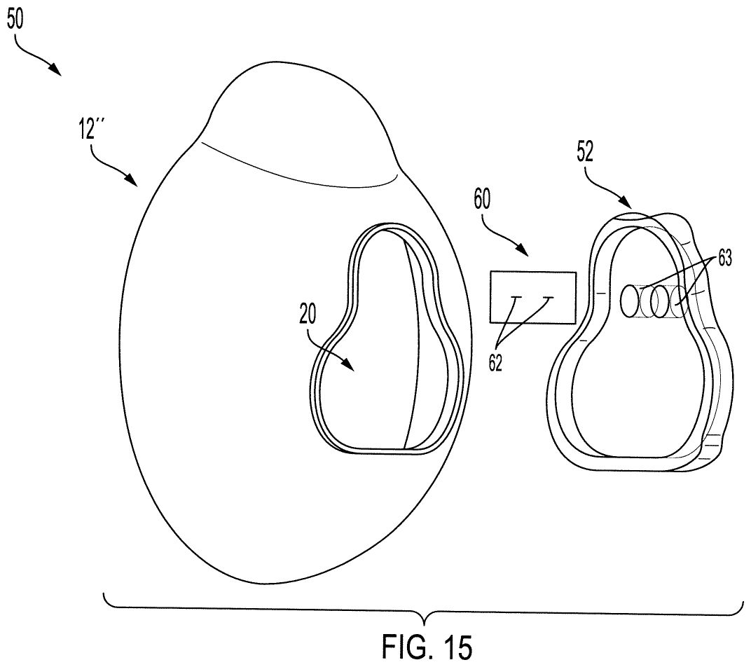

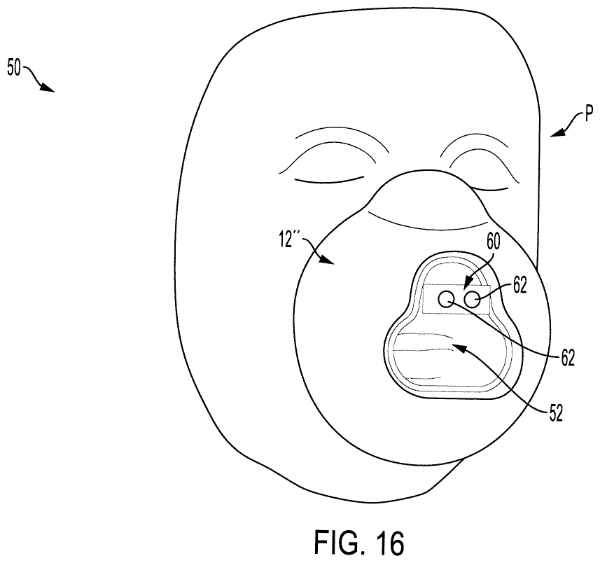

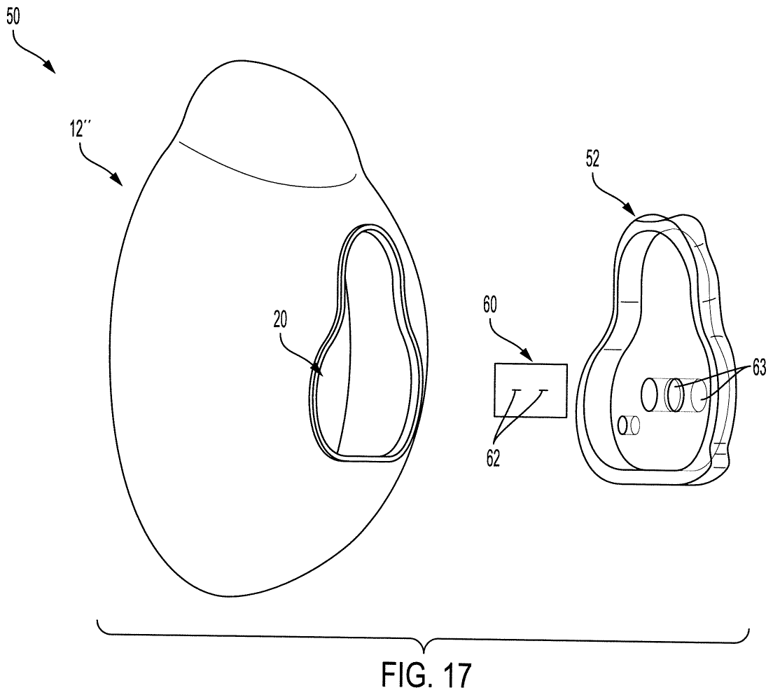

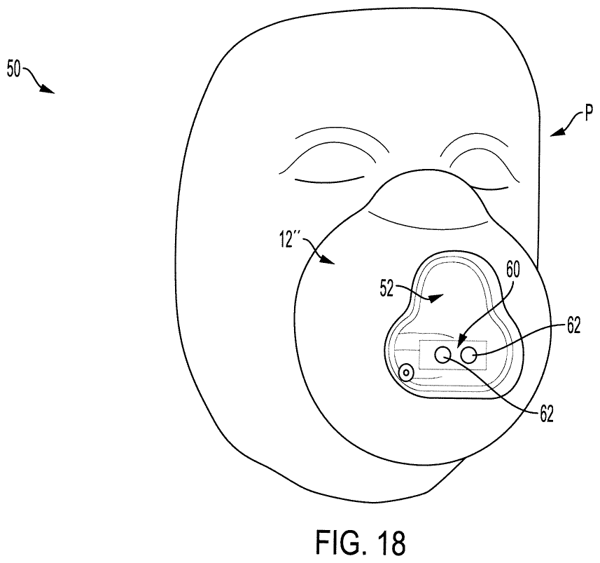

The strip 60 can be installed onto the viewing portion 52 so that the valves 62 can have a desired configuration. For example, as shown in FIGS. 15 and 16, the strip 60 can be installed onto the viewing portion 52 so that the valves 62 overlay the nostrils N (which are not shown in FIG. 16 as the nostrils N are covered by the valves 62). This configuration allows the surgical tube 8 is to be inserted into the nostrils N. In another example, as shown in FIGS. 17 and 18, the strip 60 can be installed onto the viewing portion 52 so that the valves 62 overlay the mouth M (which is not shown in FIG. 16 as the mouth M is covered by the valves 62). This configuration allows the surgical tube 8 is to be inserted into the mouth M.

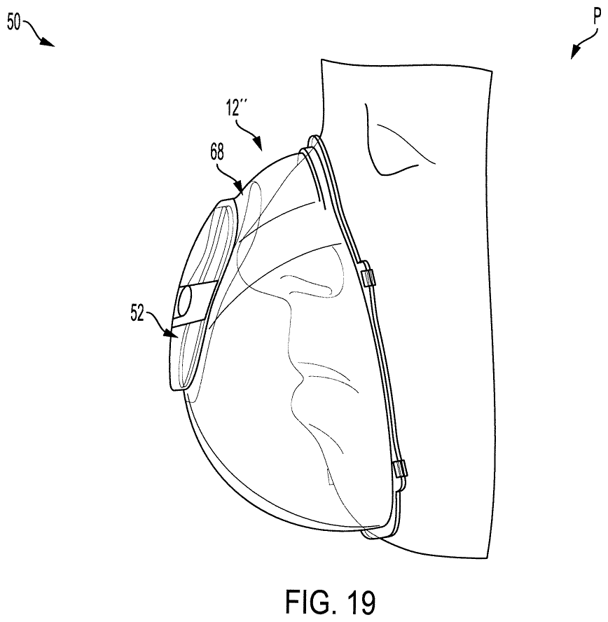

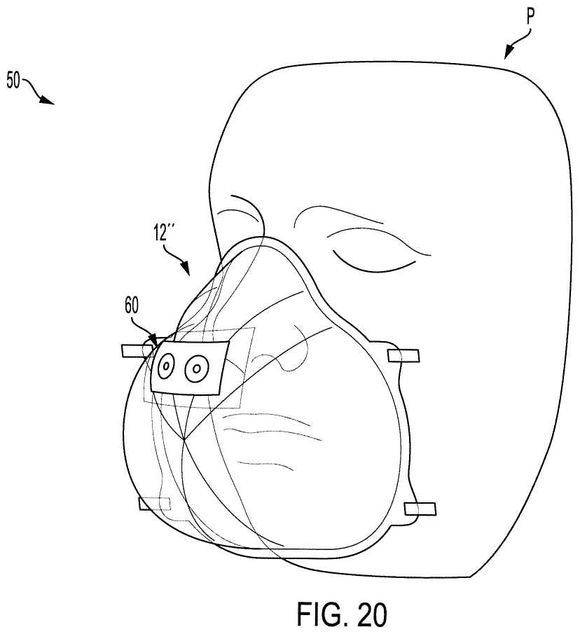

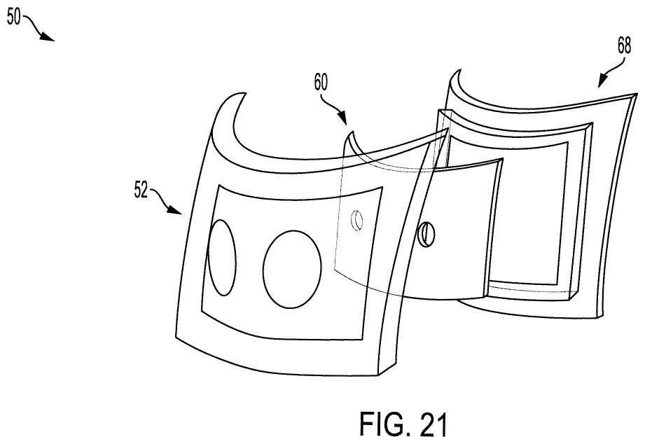

FIGS. 19-21 show other views of the medical device 50.

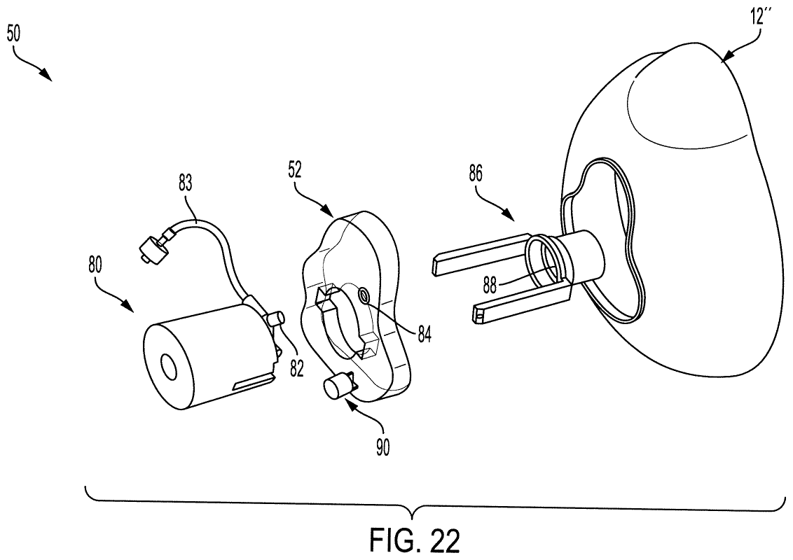

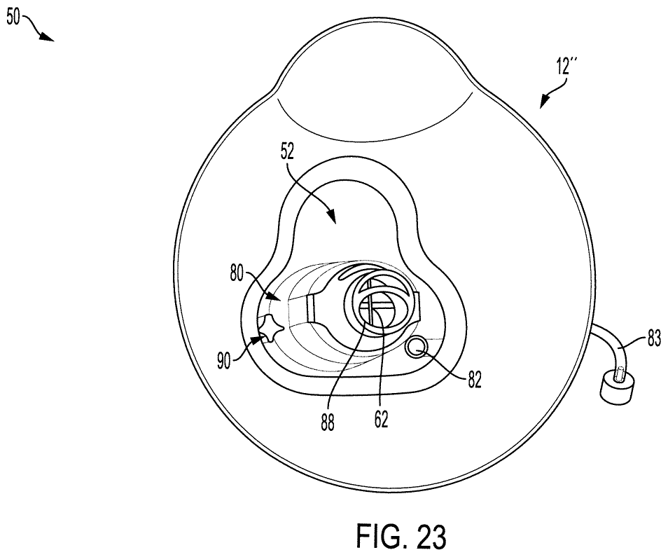

FIGS. 22 and 23 show another embodiment of the medical device 50 in use with one or more existing medical products. The medical device 50 can be connected an oxygen supply 80 via luer lock 82, The luer lock 82 fits into a corresponding opening 84 on the viewing portion 52 to attach the supply of oxygen to the medical device 50. The luer lock 82 can serve as an access point to introduce supplemental oxygen from the oxygen supply 80 to the patient P. If supplemental oxygen is not needed, a cap (not shown) can remain on the luer lock 82. If supplemental oxygen is required, the cap can be removed and oxygen tubing 83 can be screwed on to the oxygen supply 80.

In some medical procedures, such as EGD procedures or TEE procedures, a bite block 86 can be inserted into the mouth M of the patient P can required to prevent damage to the surgical tube 8 from the patient inadvertently biting down on the surgical tube 8 during conduct of the procedure. The viewing portion 52 and the backing portion 68, which is not shown in FIG. 22 for simplicity) face plate is shaped to accommodate the bite block 86. The bite clock 86 also includes an opening 88 that aligns with one of the valves 62 of the strip 60 (which is also not shown in FIG. 22 for simplicity). The bite block 86 is configured to keep the mouth M of the patient P open, while protecting the surgical tube 8. The bite block 86 can also include a valve 88 to prevent spraying of saliva from the patient's mouth M during the procedure.

In addition, an access hole or valve 90 can be provided on the viewing portion 52 to provide access for a suction device (not shown) to suction saliva from the patient's mouth M during the procedure.

FIG. 23 shows an assembled view of the medical device 50 as shown in FIG. 22.

The above description merely provides a disclosure of particular embodiments of the invention and is not intended for the purposes of limiting the same thereto. As such, the invention is not limited to only the above-described embodiments. Rather, it is recognized that one skilled in the art could conceive alternative embodiments that fall within the scope of the invention.

1-11. (canceled)

12. A medical device for use with an associated tube inserted into a respiratory outlet of a patient, the medical device comprising:
a mask including a mask opening positioned in relation to at least one respiratory outlet of a patient when the medical device is used;
a viewing portion secured to a portion of the mask and configured to overlay the mask opening, the viewing portion including at least one viewing portion opening sized to receive and allow movement of the associated tube; and
a strip releasably secured to the viewing portion, the strip including at least one valve formed in a portion thereof and configured to overlay the at least one viewing portion opening, the at least one valve being sized and dimensioned to accommodate the associated tube.

13. The medical device of claim 12, wherein the viewing portion comprises a transparent material.

14. The medical device of claim 12, wherein the at least one valve comprises a plurality of valves, the plurality of valves comprising at least:
a first valve configured to overlay a first respiratory outlet of the patient; and
a second valve configured to overlay a second respiratory outlet of the patient.

15. The medical device of claim 12, wherein the strip includes ends and the viewing portion includes slot portions configured to receive the ends of the strip.

16. The medical device of claim 12, further comprising a backing portion including:
a viewing window;
a rim; and
a protruding portion extending from the rim.

17. The medical device of claim 16, wherein
the viewing portion includes a rim and a viewing panel, the rim of the viewing portion being configured to engage the protruding portion of the rim of the backing portion; and
wherein:
the backing portion is disposed in an interior of the mask in which the protruding portion of the rim of the backing portion extends through the mask opening;
the viewing portion is attached to the mask in which the rim of the viewing portion engages the protruding portion; and
ends of the strip are inserted into corresponding slot portions of the viewing portion.

18-20. (canceled)

21. The medical device of claim 12, wherein the viewing portion is configured to allow visualization of the associated tube during insertion into the respiratory outlet of the patient.

22. A medical device for use with an associated tube inserted into a respiratory outlet of a patient and an associated mask positioned over a mouth and nostrils of the patient, the mask including a mask opening positioned in relation to the respiratory outlet of the patient when the medical device is used, the medical device comprising:
a viewing portion securable to a portion of the mask and configured to overlay the mask opening thereof; and
a strip releasably secured to the viewing portion, the strip including at least one valve formed in a portion thereof, the at least one valve being sized and dimensioned to accommodate the associated tube.

23. The medical device of claim 22, wherein the viewing portion comprises a transparent material.

24. The medical device of claim 22, wherein the at least one valve comprises a plurality of valves, the plurality of valves comprising at least:
a first valve configured to overlay a first respiratory outlet of the patient; and
a second valve configured to overlay a second respiratory outlet of the patient.

25. The medical device of claim 22, wherein the strip includes ends and the viewing portion includes slot portions configured to receive the ends of the strip.

26. The medical device of claim 22, wherein the viewing portion includes at least two viewing portion openings; and
the at least one valve of the strip comprises at least two valves that are respectively configured to overlap overlay the at least two viewing portion openings.

27. The medical device of claim 22, wherein the viewing portion is configured to allow visualization of the associated tube during insertion into the respiratory outlet of the patient.

28. The medical device of claim 22, further comprising a backing portion including:
a viewing window;
a rim; and
a protruding portion extending from the rim.

29. A medical device for use with an associated tube inserted into a respiratory outlet of a patient, the medical device comprising:
a mask including a mask opening configured to be positioned in relation to at least one respiratory outlet of a patient when the medical device is used;
a viewing portion releasably secured to a portion of the mask and configured to overlay the mask opening; and
a strip releasably secured to the viewing portion, the strip including at least one valve formed in a portion thereof, the at least one valve being sized and dimensioned to accommodate the associated tube.

30. The medical device of claim 29, wherein the viewing portion includes at least two viewing portion openings; and
the at least one valve of the strip comprises at least two valves that are respectively configured to overlay the at least two viewing portion openings.

31. The medical device of claim 29, wherein the viewing portion is configured to allow visualization of the associated tube during insertion into the respiratory outlet of the patient.

32. A medical device for use with at least one associated tube inserted into a respiratory outlet of a patient, the medical device comprising:
a mask including a main mask body with a mask opening positioned in relation to a respiratory outlet of a patient when the medical device is used, wherein the mask opening is sized to receive and allow movement of a first associated tube; and
a valve releasably secured to the main mask body and configured to overlay the mask opening, the valve being sized and dimensioned to accommodate the first associated tube.

33. The medical device of claim 32, wherein the main mask body comprises a transparent material.

34. The medical device of claim 32, the main mask body with a second mask opening positioned in relation to a second respiratory outlet of the patient when the medical device is used, wherein the second mask opening is sized to receive and allow movement of a second associated tube, the medical device further including:
a second valve releasably secured to the main mask body and configured to overlay the second mask opening, the second valve being sized and dimensioned to accommodate the second associated tube.
